(19) United States
(12) Patent Application Publication
(10) Pub. No.: US 2008/0262214 A1
(43) Pub. Date: Oct. 23, 2008

(54) Synthesis of 2-Substituted Adenosines

(75) Inventor: Edward Daniel Savory, Cambridge (GB)

Correspondence Address: FISH & RICHARDSON PC, P.O. BOX 1022, MINNEAPOLIS, MN, 55440-1022, US

(73) Assignee: CAMBRIDGE BIOTECHNOLOGY LIMITED, Cambridge (GB)

(21) Appl. No.: 10/581,544

(22) PCT Filed: Dec. 3, 2004
(86) PCT No.: PCT/GB2004/005092

§ 371 Date: Jul. 8, 2008

(30) Foreign Application Priority Data
Dec. 5, 2003 (GB) 0328323.1

(51) Int. Cl. C07H 19/167

(52) U.S. Cl. 536/27.6

(57) ABSTRACT

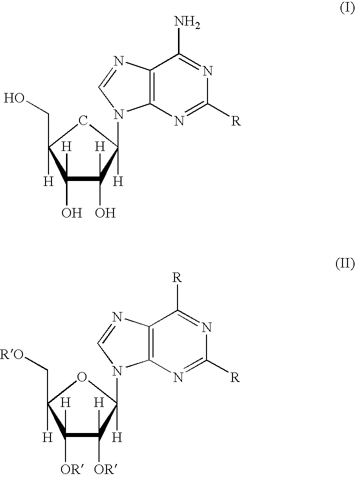

A method of synthesis of a 2-substituted adenosine of formula I which comprises converting a compound of formula II to a compound of formula (I), wherein: R is C 1-6 alkoxy (straight or branched), a phenoxy group (unsubstituted, or mono-, or di-substituted by halo, amino, CF3-, cyano, nitro, C 1-6 alkyl, or C 1-6 alkoxy), a benzoyl group (unsubstituted, or mono-, or di-substituted by halo, amino, CF3-, cyano, nitro, C1_6 alkyl, or C1_6 alkoxy), or a benzoyl group (unsubstituted, or mono-, or di-substituted by halo, amino, CF3-, cyano, nitro, C 1-6 alkyl, or C 1-6 alkoxy); R′═H, or a protecting group.

This invention relates to synthesis of 2-substituted adenosines, such as spongosine (2-methoxyadenosine), and synthesis of intermediates for use in the synthesis of such compounds.

The natural product spoagosine was first isolated from a sponge, Cryptotethia crypta, collected off the Florida coast in 1945 (Bergmann and Feeney, J. Org. Chem. 1951, 16, 981; Ibid 1956, 21, 226). Spongosine was considered an unusual nucleoside in that it was not only the first methoxypurine to be found in nature but also one of the first O-methyl compounds to be isolated from animal tissues.

Several methods of synthesis of spongosine have been reported. One of the first of these to be published was by Bergmann and Stempien (J. Org. Chem. 1957, 22, 1575) in which spongosine was formed via coupling of chloromercuric 2-methoxyadenine to 2,3,5-tri-O-benzoyl-D-ribofuranosyl chloride. This simple coupling reaction provided a crude yield of spongosine of 31% which was then recrystallised from hot water to provide spongosine which exhibited a melting point of 191-191.5° C. and an optical rotation of −43.5° (NaOH).

A variation on this theme was employed by Ojha et al. (Nucleosides and Nucleotides (1995, 14, 1889) who initially coupled 2-ethylthioadenine with a suitably protected ribose. Subsequent adjustments of the protecting groups and oxidation gave a substrate which was reacted with sodium methoxide to yield spongosine in a yield of 87% for the final step. The purity of the target spongosine after column chromatography, was proved by both elemental analysis and melting point (189-190° C.).

One of the most common methods of preparation of spongosine is via displacement of a 2-substituted chlorine atom by methoxide:

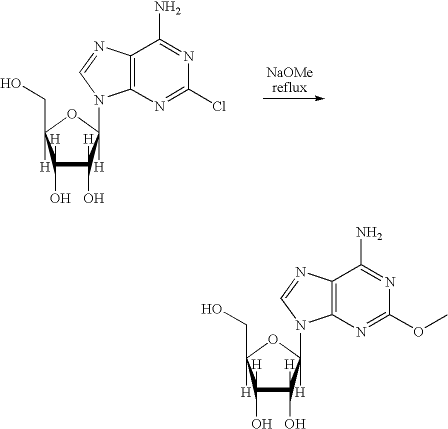

This methodology has been successfully applied by a number of groups to provide spongosine in varying yields and purity: Schaeffer et al., J. Am. Chem. Soc. 1958, 80, 3738 (35% yield, mpt. 190-192° C.); Bartlett et al., J. Med. Chem. 1981, 24, 947 (yield and purity not quoted); Sato et al., Synth. Proceed. Nucleic Acid Chem. 1968, 1, 264. However, this method suffers from the disadvantage that the 2-chloroadenosine starting material is difficult to synthesise, and consequently is expensive to produce.

Spongosine was reported by Cook et al. (J. Org. Chem. 1980, 45, 4020) as a by-product in the methylation reaction of isoguanosine by methyl iodide. Both the desired 1-methylisoguanosine and the spongosine were obtained in poor crude yields (19 and 30% respectively). The crude spongosine fragment was first purified by column chromatography on silica gel (eluent: chloroform/methanol) and then recrystallised from water to provide a sample which melted between 189-192° C. (7% yield pure).

Deghati et al (Tetrahedron Letters 41 (2000) 1291-1295) and Wanner et al (Bioorganic & Medicinal Chemistry Letters 10 (2000) 2141-2144) describe formation of spongosine as a significant by-product in the synthesis of 2-nitroadenosine by treatment of 2-nitroadenosine pentaacetate with potassium cyanide in methanol. The 2-nitroadenosine was obtained in only 10% yield, and spongosine in 47% yield (Deghati et al). The 2-nitroadenosine pentaacetate was produced by nitration of adenosine pentaacetate with tetrabutylammonium nitrate/trifluoroacetic anhydride (TBAN/TFAA):

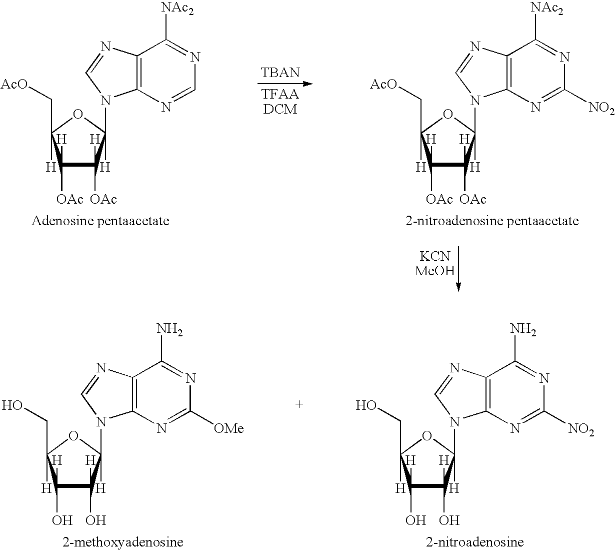

A disadvantage of this method is that the spongosine is not produced in high yield or purity. A further disadvantage is that it involves use of the toxic reagent potassium cyanide.

It is desired, therefore, to provide alternative methods of synthesis of spongosine and other 2-substituted adenosines, and of intermediates for use in the synthesis of these compounds. It is also desired to improve the yield and purity of the 2-substituted adenosines and intermediates obtained.

According to a first aspect of the invention there is provided a method of synthesis of a compound of formula I which comprises converting a compound of formula II to a compound of formula I:

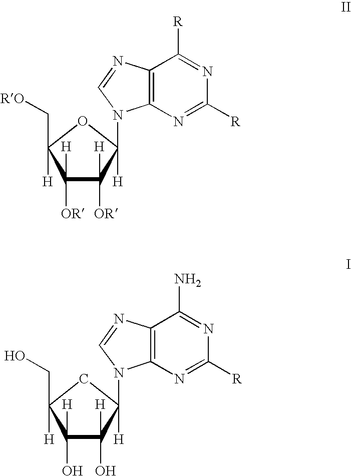

wherein:
R is C₁₋₆ alkoxy (straight or branched), a phenoxy group (unsubstituted, or mono-, or di-substituted by halo, amino, CF₃—, cyano, nitro, C₁₋₆ alkyl, or C₁₋₆ alkoxy), a benzyloxy group (unsubstituted, or mono-, or di-substituted by halo, amino, CF₃—, cyano, nitro, C₁₋₆ alkyl, or C₁₋₆ alkoxy), or a benzoyl group (unsubstituted, or mono-, or di-substituted by halo, amino, CF₃—, cyano, nitro, C₁₋₆ alkyl, or C₁₋₆ alkoxy);
R′═H, or a protecting group.

Preferably R is methoxy, ethoxy, propoxy, butoxy, pentyloxy, hexyloxy, phenoxy, benzyloxy, or benzoyl. More preferably R is methoxy.

It is preferred that the R groups of formula I are the same as each other, although in some circumstances it may be preferred that the R groups are different from one another.

It is preferred that the R′ groups are the same as each other. However, in some circumstances it may be preferred that two or three different R′ groups are used (for example one acetyl group and two benzoyl groups, or two acetyl groups and one benzoyl group).

Preferably the compound of formula I produced is isolated.

In some preferred embodiments of the invention R′ is H, and the compound of formula II is aminated to form the compound of formula I. This may be achieved, for example by heating the compound of formula I in a solution of ammonia (for example upto 80° C.) and then cooling the solution to precipitate the compound of formula I. Preferably an aqueous solution of ammonia is used, although ammonia in methanol or ethanol may alternatively be used. Preferably the precipitate is then isolated, for example by filtration and washing.

Preferably the compound of formula II is 2,6-dimethoxy adenosine, and the compound of formula I is spongosine. A preferred method of converting 2,6-dimethoxy adenosine to spongosine and isolating the spongosine produced is described in Step 5 of the Example below.

In other preferred embodiments of the invention R′ is a protecting group. It is advantageous if the protecting group is removed under the same conditions that replace the R group at the 6-position of the purine component of the compound of formula I with an amino group. This allows the compound of formula II to be converted to the compound of formula I in a single reaction step. It is preferred that R′ is a protecting group that can be removed by treatment with ammonia. Suitable protecting groups are acetyl and benzoyl.

Preferably methods of the first aspect of the invention further comprise converting a compound of formula II (preferably triacetoxy 2-nitro-6-chloroadenosine) to a compound of formula II:

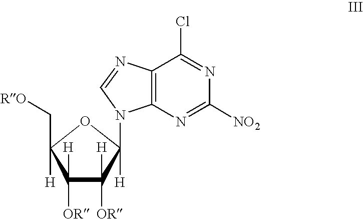

wherein R″ is a protecting group, preferably acetyl or benzoyl.

It is preferred that the R″ protecting groups are the same as each other. However, in some circumstances it may be preferred that two or three different R″ protecting groups are used (for example one acetyl group and two benzoyl groups, or two acetyl groups and one benzoyl group).

According to a further aspect of the invention there is provided a method of synthesis of a compound of formula I which includes the step of converting a compound of formula III (preferably triacetoxy 2-nitro-6-chloroadenosine) to a compound of formula II.

There is also provided according to a further aspect of the invention a method of synthesis of a compound of formula II which comprises converting a compound of formula III (preferably triacetoxy 2-nitro-6-chloroadenosine) to the compound of formula II.

Preferably the compound of formula II produced is isolated.

When the R′ groups of the compound of formula II are protecting groups, it will be appreciated that they will usually be the same as each other, and the same as the R″ protecting groups of the compound of formula III. However, in some circumstances it may be desired that the R″ protecting groups are different to the R′ protecting groups.

Preferably the compound of formula III (for example triacetoxy 2-nitro-6-chloroadenosine) is alkoxylated or benzoylated at the 2- and 6-positions to form the compound of formula II.

Preferably the compound of formula I is spongosine, and the compound of formula II is 2,6-dimethoxy adenosine.

For embodiments of the invention in which the compound of formula I is 2,6-dimethoxyadenosine, and the compound of formula III is triacetoxy 2-nitro-6-chloroadensine, preferably the triacetoxy 2-nitro-6-chloroadensine is methoxylated at the 2- and 6-positions to form 2,6-dimethoxy adenosine. This may be achieved, for example by contacting a solution of sodium methoxide in methanol with a solution of triacetoxy 2-nitro-6-chloroadenosine in dichloromethane (DCM) or chloroform.

An advantage of use of sodium methoxide/methanol as methoxylating reagent is that it is considerably less toxic than potassium cyanide/methanol used by Deghati et al., and Wanner et al. Sodium methoxide/methanol also appears to give a higher yield of methoxylated product than potassium cyanide/methanol.

Preferably the 2,6-dimethoxy adenosine is then isolated from the contacted solutions, for F example by removing the methanol and DCM and purifying the 2,6-dimethoxy adenosine by reverse phase column chromatography.

A preferred method of converting triacetoxy 2-nitro-6-chloroadenosine to 2,6-dimethoxy adenosine and isolating the 2,6-dimethoxyadenosine produced is described in Step 4 of the Example below.

Preferably methods of the first or further aspects of the invention further comprise converting a compound of formula IV (preferably triacetoxy 6-chloroadenosine) to a compound of formula I (preferably triacetoxy 2-nitro-6-chloroadenosine):

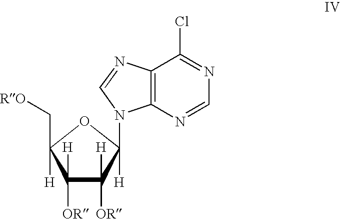

wherein R″ is a protecting group, preferably acetyl or benzoyl. The R″ protecting groups should preferably be the same as the R″ protecting groups of formula III.

According to a further aspect of the invention there is provided a method of synthesis of a compound of formula I or a compound of formula I which includes the step of converting a compound of formula IV (preferably triacetoxy 6-chloroadenosine) to a compound of formula II (preferably triacetoxy 2-nitro-6-chloroadenosine).

Preferably the compound of formula III (for example triacetoxy 2-nitro-6-chloroadenosine) produced is isolated.

Preferably the compound of formula I is spongosine, and the compound of formula II is 2,6-dimethoxy adenosine.

Preferably triacetoxy 6-chloroadenosine is nitrated at the 2-position to form triacetoxy 2-nitro, 6-chloroadenosine. Suitable nitrating reagents include tetrabutyl ammonium nitrate (TBAN), tetramethyl ammonium nitrate (TMAN) and sodium nitrate. For example a solution of triacetoxy 6-chloroadenosine may be contacted with a solution of TBAN and trifluoroacetic acid (TFAA), or TMAN and TFAA. Preferably a chlorinated solvent is used, such as DCM or chloroform.

Nitration of triacetoxy 6-chloroadenosine to triacetoxy 2-nitro-6-chloroadenosine using TBAN/TFAA in DCM is described in Deghati et al., page 1292, lines 4-6 (although not in relation to synthesis of spongosine). TBAN/TFAA is also used by Deghati et al. to nitrate adenosine pentaacetate in the method of synthesis of spongosine disclosed in this document.

We have appreciated, however, that one of the principal reasons that spongosine is not produced in high yield and purity by the method of Deghati et al. is that TBAN and other tetrabutyl ammonium (TBA) salts contaminate the 2-nitroadenosine pentaacetate intermediate and interfere with subsequent synthesis steps.

According to the invention, the yield and purity of the spongosine product can be significantly improved if the amount of contaminating TBA salts is reduced. However, removal of these contaminants is problematic because they are amphiphilic and so cannot be completely removed by aqueous; work-up.

We have found that the purity and yield of triacetoxy 2-nitro-6-chloroadenosine and subsequently produced 2,6-dimethoxyadenosine and spongosine is surprisingly significantly improved by trituration of the triacetoxy 2-nitro-6-chloroadenosine from isopropanol, or preferably ethanol, and washing with a mixture of water and ethanol to remove the TBA impurities.

We have appreciated that similar methods can be used to remove tetramethyl ammonium (TMA) impurities if tetramethyl ammonium nitrate (TMAN) is used as nitrating reagent instead of TBAN. Use of TMAN as nitrating agent may be preferred to use of TBAN because TMAN is easier to wash out with water than TBAN.

The TBA or TMA impurities are easier to remove from triacetoxy 2-nitro-6-chloroadenosine than from 2-nitroadenosine pentaacetate (used by Deghati et al.) because this latter compound decomposes in water. Thus, spongosine can be synthesised more easily in high yield and purity by using a triacetoxy 6-chloroadenosine intermediate.

A preferred method of converting triacetoxy 6-chloroadenosine to triacetoxy 2-nitro-6-chloroadenosine and isolating the triacetoxy 2-nitro-6-chloroadenosine produced is described in Step 3 of the Example below.

We have appreciated that the above methods can be used to remove TBA or TMA impurities that contaminate compounds synthesised in other reactions by nitration of a substituted adenosine using TBAN or TMAN. The compounds may thereby be produced in higher purity, and the purity and yield of products produced by subsequent synthesis steps may be increased.

Thus, according to a further aspect of the invention there is provided a method of reducing the amount of TBA or TMA impurities contaminating a product formed by nitration of a substituted adenosine with TBAN or TMAN, which comprises triturating the product from isopropanol or ethanol, and washing the product with a mixture of water and ethanol.

There is also provided according to the invention a method of producing a nitrated substituted adenosine which comprises nitrating a substituted adenosine using TBAN or TMAN, and reducing the amount of TBA or TMA impurity contaminating the nitrated substituted adenosine.

Preferably the substituted adenosine is a compound of formula VI:

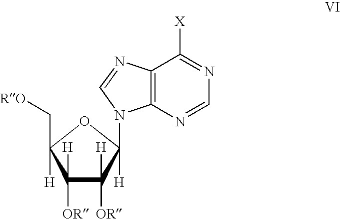

wherein:
X is halo, preferably Cl, or —OMe; and
R″ is H, or a protecting group, preferably acetyl or benzoyl.

Preferably the amount of TBA or TMA impurity is reduced by triturating the nitrated substituted adenosine from isopropanol or ethanol, and washing the triturated product with a mixture of water and ethanol.

In general, a minimum of three washes with water/ethanol has been found to be required to remove a large proportion of the TBA or TMA impurities. However, five washes are generally carried out to ensure as much TBA or TMA impurity is removed as possible.

Instead of trituration, it may be possible to use column chromatography or reverse phase chromatography to reduce the amount of TBA or TMA impurity present.

The invention also provides nitrated, substituted adenosines produced by such methods.

Preferably methods of the first or further aspects of the invention further comprise converting a compound of formula V (preferably triacetoxy inosine) to a compound of formula IV (preferably triacetoxy 6-chloro adeno sine):

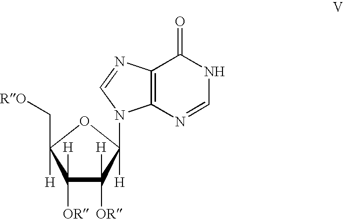

wherein R″ is a protecting group, preferably acetyl or benzoyl. The R″ protecting groups should preferably be the same as the R″ protecting groups of formula IV (and/or formula III).

According to a further aspect of the invention there is provided a method of synthesis of a compound of formula I, a compound of formula II, or a compound of formula III, which includes the step of converting a compound of formula V (preferably triacetoxy inosine) to a compound of formula IV (preferably triacetoxy 6-chloroadenosine).

Preferably the compound of formula IV (for example triacetoxy 6-chloroadenosine) produced is isolated.

Preferably the compound of formula I is spongosine, and the compound of formula II is 2,6-dimethoxy adenosine.

Preferably triacetoxy inosine is chlorinated to form triacetoxy 6-chloroadenosine. This may be achieved, for example by contacting ID and thionyl chloride with a solution of triacetoxy inosine in chloroform. Instead of chloroform, DCM may be used as a solvent. Instead of thionyl chloride, POCl₃ may be used as chlorinating reagent.

Preferably the triacetoxy 6-chloroadenosine is isolated from the contacted DMF, thionyl chloride, and triacetoxy inosine solution, for example by removal of the DMF, thionyl chloride, and chloroform, partitioning of the resulting residue between DCM and aqueous sodium bicarbonate, and washing of the separated organic phase with brine and drying over magnesium sulphate.

A preferred method of forming the triacetoxy 6-chloroadenosine from triacetoxy inosine, and isolating the triacetoxy 6-chloroadenosine produced is described in step 2 of the Example below.

Preferably methods of the first or further aspects of the invention further comprise converting inosine to a compound of formula V (preferably triacetoxy inosine).

According to a further aspect of the invention there is provided a method of synthesis of a compound of formula I, II, III, or IV, which includes the step of converting inosine to a compound of formula V (preferably triacetoxy inosine).

Preferably the compound of formula V (for example triacetoxy inosine) produced is isolated.

Preferably the compound of formula I is spongosine, and the compound of formula II is 2,6-dimethoxy adenosine.

Preferably inosine is acetylated or benzoylated to form the compound of formula V (preferably triacetoxy inosine). Acetylation of inosine to form triacetoxy inosine may be achieved, for example by contacting a suspension of inosine and catalytic DMAP in MeCN with Et₃N and acetic anhydride to form a solution before contacting the solution with methanol.

A preferred method of converting inosine to triacetoxy inosine and isolating the triacetoxy inosine produced is described in Step 1 of the Example below.

According to the invention there is also provided use of a compound of formula II, III (preferably triacetoxy 2-nitro, 6-chloroadenosine), IV (preferably triacetoxy 6-chloroadenosine), V (preferably triacetoxy inosine), or inosine in the synthesis of a compound of formula I.

The invention further provides use of a compound of formula II (preferably triacetoxy 2-nitro, 6-chloroadenosine), IV (preferably triacetoxy 6-chloroadenosine), V (preferably triacetoxy inosine), or inosine in the synthesis of a compound of formula II.

Preferably the compound of formula I is spongosine and the compound of formula II is 2,6-dimethoxy adenosine.

Methods of the invention allow synthesis of 2-substituted adenosines and intermediates for use in the synthesis of 2-substituted adenosines in high yield and purity, and do not require use of toxic reagents such as potassium cyanide.

Embodiments of the invention are now described by way of example only with reference to the accompanying Scheme 1 which shows the synthesis of spongosine from inosine.

### EXAMPLE

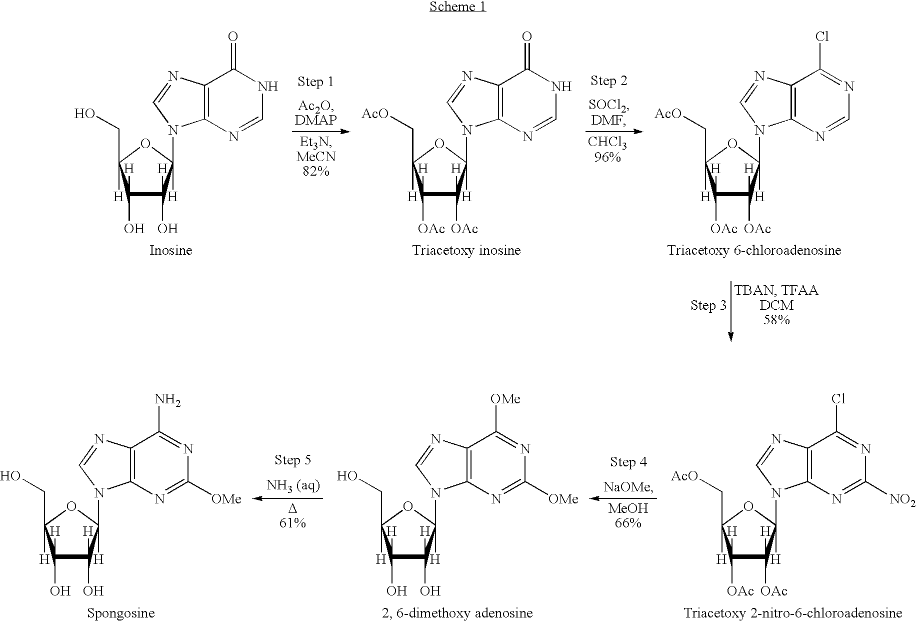

### Step 1

To a suspension of inosine (10 g, 37.3 mmol) and catalytic DMAP in MeCN (60 mL) was added Et₃N (20 mL, 143 mmol) and acetic anhydride (12.5 mL) and the resulting solution was stirred for 1 h at ambient temperature before the addition of MeOH (5 mL). After stirring for 5 mins, the solution was concentrated in vacuo to yield a white solid which was washed with isopropyl alcohol to afford triacetoxy inosine (12.1 g, 82%).

To a solution of triacetoxy inosine (3.00 g, 7.63 mol) in CHCl₃ (25 mL) was added DMF (1.80 mL, 22.9 mmol) and thionyl chloride (1.68 mL, 22.9 mol) and the resulting solution was refluxed overnight before removal of the solvents in vacuo. The residue was then partitioned between DCM and aq. NaHCO₃ and the separated organic phase was washed with brine and dried over MgSO₄ to afford triacetoxy 6-chloroadenosine as a pale yellow oil (3.03 g, 96%).

### Step 3

To a solution of TBAN (4.43 g, 14.5 mmol) in DCM (15 mL) at 0° C. was added TFAA (2.05 mL, 14.5 mmol) and the resulting solution was stirred for 5 mins, before the addition of triacetoxy 6-chloroadenosine (4 g, 9.7 mmol) in DCM (2 mL). The resulting brown solution was stirred for 2.5 h before being quenched with aq. NaHCO₃, extracted into DCM and dried over MgSO₄. Purification via trituration from EtOH yielded triacetoxy 2-nitro, 6-chloroadenosine as a pale yellow solid which was washed with 1:1 EtOH/water to afford 2.57 g, 58%.

### Step 4

To a solution of NaOMe (590 mg, 10.9 mmol) in MeOH (10 mL) was added dropwise a solution of triacetoxy 2-nitro, 6-chloroadenosine (1 g, 2.19 mmol) in DCM (10 mL) and the resulting red solution was stirred overnight. The solvents were then removed in vacuo and the product was purified by reverse phase column chromatography (gradient 30-70% MeOH/water) to afford 2,6-dimethoxy adenosine as a white solid (447 mg, 66%).

### Step 5

A solution of 2,6-dimethoxy adenosine (697 mg, 3.23 mmol) in aq. NH₃ was heated in a sealed tube at 80° C. for 26 h. The solution was then cooled and the resulting white precipitate was filtered and washed with cold water to afford 2-methoxy adenosine (406 mg, 61%).

In other preferred embodiments benzoyl protecting groups may be used instead of the acetyl protecting groups shown.

1. A method of synthesis of a 2-substituted adenosine of formula I which comprises converting a compound of formula II to a compound of formula I: 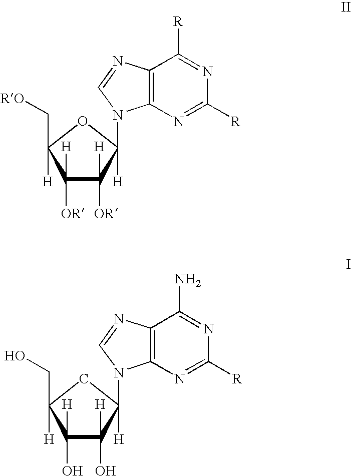
wherein:
R is C₁₋₆ alkoxy (straight or branched), a phenoxy group (unsubstituted, or mono-, or di-substituted by halo, amino, CF₃—, cyano, nitro, C₁₋₆ alkyl, or C₁₋₆ alkoxy), a benzyloxy group (unsubstituted, or mono-, or di-substituted by halo, amino, CF₃—, cyano, nitro, C₁₋₆ alkyl, or C₁₋₆ alkoxy), or a benzoyl group (unsubstituted, or mono-, or di-substituted by halo, amino, CF₃—, cyano, nitro, C₁₋₆ alkyl, or C₁₋₆ alkoxy);
R′═H, or a protecting group.

2. A method according to claim 1, wherein R=methoxy, ethoxy, propoxy, butoxy, pentyloxy, hexyloxy, phenoxy, benzyloxy, or benzoyl.

3. A method according to claim 1, wherein R′ is a protecting group that can be removed under conditions that replace the R group with an amino group at the 6-position of the purine component of the compound of formula II.

4. A method according to claim 3, wherein the compound of formula II is converted to the compound of formula I in a single reaction step.

5. A method according to claim 1, wherein the protecting group is acetyl or benzoyl, and the compound of formula II is converted to the compound of formula I by treatment with ammonia.

6. A method according to claim 1, wherein R′ is H, and the compound of formula II is aminated to form the compound of formula I.

7. A method according to claim 6, wherein the compound of formula II is, aminated by heating the compound in a solution of ammonia and then cooling the solution to precipitate the compound of formula I.

8. A method according to claim 1, which further comprises isolating the compound of formula I produced.

9. A method according to claim 1, which further comprises converting a compound of formula III to a compound of formula II: 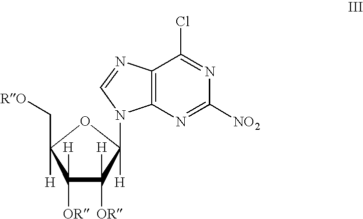
wherein R″ is a protecting group, preferably acetyl or benzoyl.

10. A method of synthesis of a compound of formula II which comprises converting a compound of formula III to the compound of formula II.

11. A method according to claim 9, wherein the compound of formula III is alkoxylated or benzoylated to form the compound of formula II.

12. A method according to claim 9, wherein the compound of formula III is triacetoxy 2-nitro-6-chloroadenosine.

13. A method according to claim 12, wherein triacetoxy 2-nitro-6-chloroadenosine is methoxylated using sodium methoxide in methanol as methoxylating reagent.

14. A method according to claim 9, which further comprises isolating the compound of formula II produced.

15. A method according to claim 9, which further comprises converting a compound of formula IV to a compound of formula III: 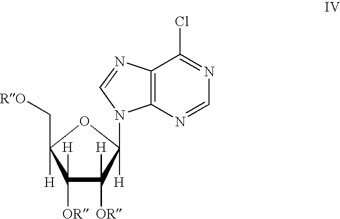
wherein R″ is a protecting group, preferably acetyl or benzoyl.

16. A method according to claim 15, wherein the compound of formula IV is nitrated to form the compound of formula III.

17. A method according to claim 15, which further comprises isolating the compound of formula III produced.

18. A method according to claim 15, wherein the compound of formula IV is triacetoxy 6-chloroadenosine, and the compound of formula III is triacetoxy 2-nitro-6-chloroadenosine.

19. A method according to claim 18, wherein triacetoxy 6-chloroadenosine is nitrated to triacetoxy 2-nitro-6-chloroadenosine using tetrabutyl ammonium nitrate (TBAN) or tetramethyl ammonium nitrate (TMAN) as nitrating reagent.

20. A method according to claim 19, which further comprises reducing the amount of tetrabutyl ammonium (TBA) or tetramethyl ammonium (TMA) impurities contaminating the triacetoxy 2-nitro-6-chloroadenosine.

21. A method according to claim 20, wherein the amount of TBA or TMA impurities is reduced by triturating the triacetoxy 2-nitro-6-chloroadenosine from isopropanol or ethanol, and washing the triturated triacetoxy 2-nitro-6-chloroadenosine with a mixture of water and ethanol.

22. A method according to claim 15, which further comprises converting a compound of formula V to a compound of formula IV: 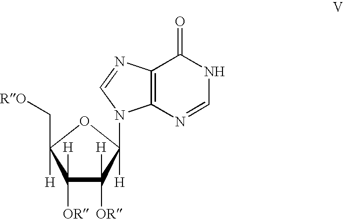
wherein R″ is a protecting group, preferably acetyl or benzoyl.

23. A method according to claim 22, wherein the compound of formula V is chlorinated to form the compound of formula IV.

24. A method according to claim 22, wherein the compound of formula V is triacetoxy inosine, and the compound of formula IV is triacetoxy 6-chloroadenosine.

25. A method according to claim 24, wherein triacetoxy inosine is chlorinated using thionyl chloride or POCl₃ as chlorinating reagent.

26. A method according to claim 22, which further comprises isolating the compound of formula IV produced.

27. A method according to claim 22, which further comprises converting inosine to a compound of formula V.

28. A method according to claim 27, wherein inosine is acetylated or benzoylated to form the compound of formula V.

29. A method according to claim 27, wherein the compound of formula V is triacetoxy inosine.

30. A method according to claim 29, wherein inosine is acetylated using acetic anhydride as acetylating reagent.

31. A method according to claim 27, which further comprises isolating the compound of formula V produced.

32. A method of synthesis of spongosine which comprises the steps shown in scheme 1.

33. A method of synthesis of spongosine which is substantially as described with reference to steps 1 to 5 of the Example.

34. A 2-substituted adenosine of formula I synthesized by a method according to claim 1.

35. A method of synthesis of 2,6-dimethoxy adenosine which is substantially as described with reference to steps 1 to 4 of the Example.

36. A compound of formula II synthesized by a method according to claim 10.

37. Use of a compound of formula II, III, IV, V, triacetoxy 2-nitro, 6-chloroadenosine, triacetoxy 6-chloroadenosine, triacetoxy inosine, or inosine in the synthesis of a compound of formula I.

38. Use of a compound of formula III, IV, V, triacetoxy 2-nitro, 6-chloroadenosine, triacetoxy 6-chloroadenosine, triacetoxy inosine, or inosine in the synthesis of a compound of formula II.

39. A method of producing a nitrated substituted adenosine which comprises nitrating a substituted adenosine using TBAN or TMAN, and reducing the amount of TBA or TMA impurity contaminating the nitrated substituted adenosine.

40. A method according to claim 39, wherein the amount of TBA or TMA impurity is reduced by triturating the nitrated substituted adenosine from isopropanol or ethanol, and washing the triturated product with a mixture of water and ethanol.
